(19) United States
(12) Patent Application Publication
(10) Pub. No.: US 2008/0125737 A1
(43) Pub. Date: May 29, 2008

(54) UNDERGARMENT INCORPORATING INTEGRATED SANITARY NAPKIN

(76) Inventors: Mehri Modgeddi, Beverly Hills, CA (US); Negar Modgeddi, Beverly Hills, CA (US)

Correspondence Address: HECKER & HARRIMAN, SUITE 2300, 1925 CENTURY PARK EAST, LOS ANGELES, CA, 90067, US

(21) Appl. No.: 11/873,778

(22) Filed: Oct. 17, 2007

Related U.S. Application Data
(60) Provisional application No. 60/852,255, filed on Oct. 17, 2006.

(51) Int. Cl. A61F 13/15; A41B 9/04

(52) U.S. Cl. 604/385.01; 604/386; 604/396; 604/393; 2/406

(57) ABSTRACT

The present invention comprises underwear incorporating a sanitary pad. In one or more embodiments, the sanitary pad is made of a cotton-like material with velcro straps that can adjust the fit of the underwear. The pad is nestled in the crotch of the underwear with the cotton-like material encasing it. The pad preferably cannot be removed from the undergarment as it is sewn inside with the cotton-like material. The velcro straps are adjustable to fit most anyone. In one or more embodiments, the pad is intended to be disposable and can be disposed of in a trash bin after use. In one or more embodiments, the underwear is not intended to be re-used after the pad has been soiled and it is not washable. In one or more other embodiments, the undergarment and pad are both made of washable materials and may be washed and reused.

### REFERENCE TO RELATED APPLICATION

This patent application claims the benefit of U.S. Provisional Patent Application Ser. No. 60/852,255 filed Oct. 17, 2006.

### FIELD OF THE INVENTION

The invention relates to women's undergarments and sanitary napkins.

A portion of the disclosure of this patent document contains material which is subject to copyright protection. The copyright owner has no objection to the facsimile reproduction by anyone of the patent document or the patent disclosure, as it appears in the Patent and Trademark Office file or records, but otherwise reserves all copyrights associated with this document.

### BACKGROUND

Sanitary napkins are commonly used to absorb menstrual flow. They are typically worn between the body and underwear. Sanitary napkins come in a variety of absorbencies, styles, materials, and shapes.

One disadvantage with sanitary napkins is that occasionally a portion of the menstrual flow escapes the napkin and impinges on the underwear, either because the napkin shifts position, is not sufficiently absorbent, or for other reasons, causing staining and requiring additional laundering.

The present invention overcomes the limitation of the prior art by providing an undergarment with an integrated sanitary napkin. In one embodiment, the undergarment of the present invention is intended to be disposable. In another embodiment the undergarment is washable and reusable.

### SUMMARY OF THE INVENTION

The present invention comprises underwear incorporating a sanitary pad. In one or more embodiments, the undergarment is made of a cotton-like material with velcro straps that can adjust the fit of the underwear. The pad is nestled in the crotch of the underwear with the cotton-like material encasing it. The pad preferably cannot be removed from the undergarment as it is sewn inside with the cotton-like material. The velcro straps are adjustable to fit most anyone. In one or more embodiments, the pad is intended to be disposable and can be disposed of in a trash bin after use. In one or more embodiments, the underwear is not intended to be re-used after the pad has been soiled and it is not washable. In one or more other embodiments, the undergarment and pad are both made of washable materials and may be washed and reused.

### BRIEF DESCRIPTION OF THE DRAWINGS

FIG. 1 shows an example of an embodiment of the undergarment of the present invention.

FIG. 2 shows an example of an embodiment of the undergarment of the present invention.

FIG. 3 shows an example of an embodiment of the undergarment of the present invention.

FIG. 4 shows an example of an embodiment of the undergarment of the present invention.

### DETAILED DESCRIPTION OF THE INVENTION

The invention comprises a women's undergarment, for example a panty, that incorporates an integrated sanitary absorbent pad. In the following description, numerous specific details are set forth to provide a thorough description of the present invention. It will be apparent, however, to one skilled in the art, that the invention may be practiced without these specific details. In other instances, well known features have not been described in detail so as not to obscure the invention.

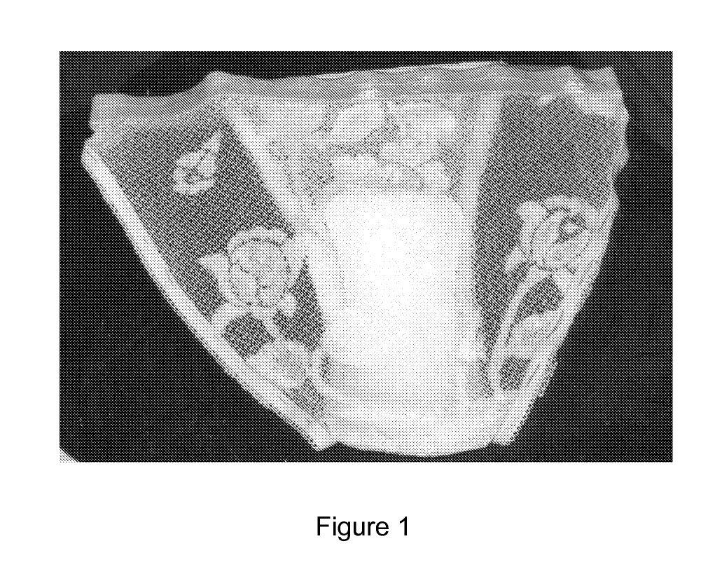

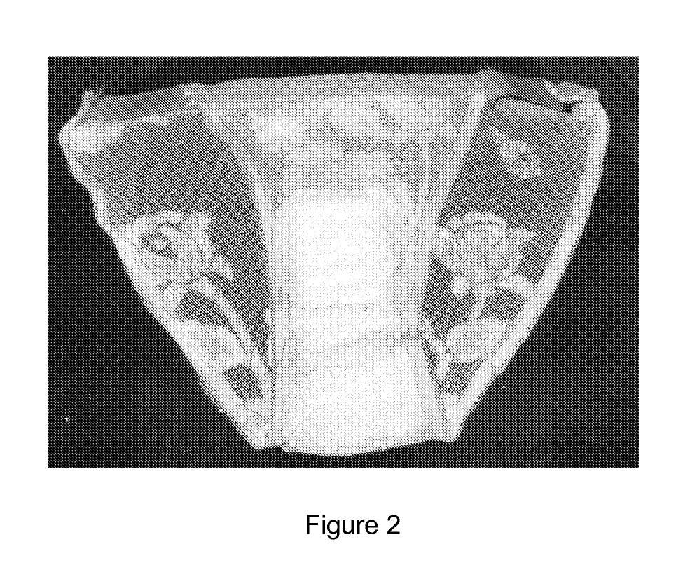

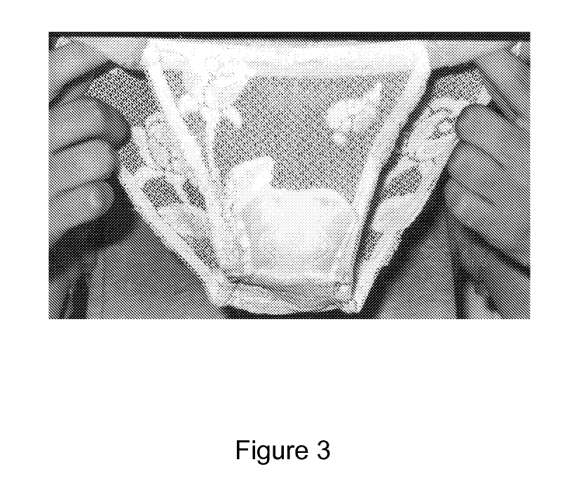

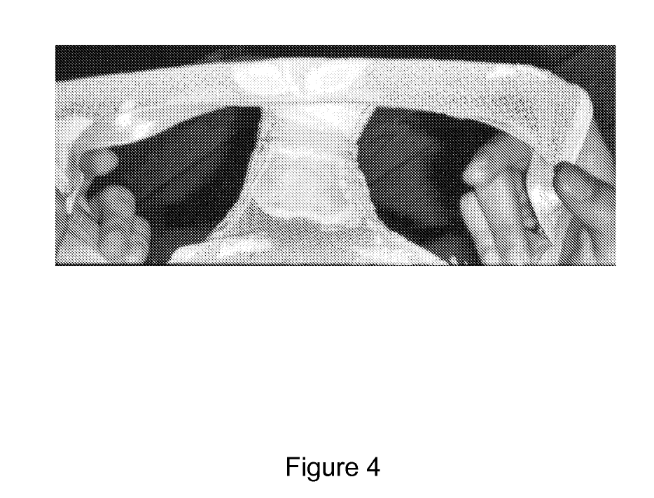

FIGS. 1-4 below show an example of an embodiment of an undergarment of the present invention.

The example embodiment of FIGS. 1-4 is made of a cotton-like material with velcro straps that can adjust the fit of the underwear. The pad is nestled in the crotch of the underwear with the cotton-like material encasing it. The pad preferably cannot be removed from the undergarment as it is sewn inside with the cotton-like material. The velcro straps are adjustable to fit most anyone. The embodiment of FIGS. 1-4 is intended to be disposable and can be disposed of in a trash bin after use. In one or more embodiments, the underwear is not intended to be re-used after the pad has been soiled and it is not washable. In one or more other embodiments, the undergarment and pad are both made of washable materials and may be washed and reused.

In one or more embodiments, the underwear of the present invention will be available in a variety of colors to match the outfit that is being worn. The pad inside the undergarment will come in two forms one being made for lighter days (a thinner pad) the other for heavier flow days (a thicker pad). The underwear itself is designed to be fashionable and sexy not like a diaper and the cut is feminine.

In one or more embodiments, the fabric of the underwear is either Modal or a cotton-like material. The material enclosing the pad is preferably very absorbent so that not only the pad but also the material is extremely absorbent. Any materials for underwear as are known to those of skill in the art may be used for the underwear, and any materials for sanitary napkins and/or absorbent pads as are known to those of skill in the art may be used for the sanitary pad.

In one or more embodiments of the invention, the undergarments are packaged a clear plastic container that allows the product to be seen from the outside. The package may be either cylindrical or square and may have the name of the product printed on the outside. The back of the box may include a description of the product and the colors available in each particular box. In one or more embodiments, a small plastic gem-like stone is attached to the outside of the box where the box is to opened. In one or more embodiments, approximately 24 panties are packaged in each package.

In one or more embodiments, the panties of the present invention may be made in a variety of styles, sizes and colors. In one or more embodiments, the colors are white, black, beige, and pink. In one or more embodiments, the sizes are small, medium and large.

Thus a novel undergarment incorporating an integrated sanitary napkin has been presented. Although the present invention has been described with respect to particular example embodiments, it will be understood by those of skill in the art that the invention is not limited to those particular embodiments, but includes alternative embodiments that will be evident to those skilled in the art.

1. An undergarment comprising:
a sanitary pad incorporated in said undergarment;
adjustable straps allowing the size of the undergarment to be varied to accommodate a plurality of body sizes.
